(19) United States
(12) Patent Application Publication
(10) Pub. No.: US 2021/0317415 A1
(43) Pub. Date: Oct. 14, 2021

(54) PERFUSION SYSTEM FOR HARVESTING BONE MARROW

(71) Applicant: Ossium Health, Inc., San Francisco, CA (US)

(72) Inventors: Erik J. WOODS, Carmel, IN (US); Brian H. JOHNSTONE, Fishers, IN (US)

(21) Appl. No.: 17/227,217

(22) Filed: Apr. 9, 2021

Related U.S. Application Data
(60) Provisional application No. 63/008,008, filed on Apr. 10, 2020.

(51) Int. Cl. C12N 5/077; C12N 5/0775; C12N 5/0789

(52) U.S. Cl. CPC C12N 5/0669; C12N 5/0647; C12N 5/0663

(57) ABSTRACT

Provided herein are methods and systems for recovering bone marrow. The methods comprising perfusing a bone with a perfusion media and recovering the liberated bone marrow cells.

### CROSS-REFERENCE TO RELATED APPLICATIONS

This application claims priority to U.S. Provisional Application No. 63/008,008 filed Apr. 10, 2020. The entire disclosure of which is are expressly incorporated herein by reference.

### STATEMENT AS TO FEDERALLY SPONSORED RESEARCH

This invention was made with government support under AI129444, AI138334, and HL142418 awarded by the National Institutes of Health. The government has certain rights in the invention.

### BACKGROUND

Bone marrow for clinical purposes is typically harvested from HLA (human leukocyte antigen) matched siblings or optimally matched unrelated donors (MUD). Other graft sources include mismatched haplo-identical related or unrelated donors, umbilical cord blood (CB), and Peripheral blood stem cells (PBSCs). When transplanted into patients with certain diseases, the hematopoietic stem cells (HSCs) in the donor bone marrow engraft in the patient and reconstitute immune and hematopoietic systems.

Bone marrow is also a source for mesenchymal stromal/stem cells (MSCs) which are self-renewing, multipotent progenitor cells with multilineage potential to differentiate into cell types of mesodermal origin, such as adipocytes, osteocytes, and chondrocytes. In addition, MSCs can migrate to sites of inflammation and exert potent immunosuppressive and anti-inflammatory effects through interactions between lymphocytes associated with both the innate and adaptive immune system.

According to current techniques of obtaining bone marrow cells from living donors, bone marrow is collected by first creating a hole in the outer bone with a trocar needle and then using a bone marrow aspiration needle and a syringe to draw the marrow into the barrel of the syringe. The syringe is then removed from the sterile field and connected to a collection bag containing anticoagulants. The bone marrow is pushed into the bag by depressing the syringe plunger. This step is repeated many times, typically in both pelvic bones, and can result in contamination of the aspirate. There remains an unmet need for methods for recovering liberated bone marrow cells with a reduced likelihood of contamination.

### SUMMARY OF THE INVENTION

An aspect of the present disclosure is a method for recovering bone marrow cells. The method comprising steps of: creating a first opening at a first surface of a bone comprising bone marrow and creating a second opening at a second surface of the bone; inserting a first cannula through the first opening and inserting a second cannula through the second opening, wherein the first cannula and second cannula gain contact with the interior of the bone; attaching a first tubing to the first cannula and a second tubing to the second cannula; providing a perfusion buffer through the first tubing, thereby perfusing the interior of the bone with the perfusion buffer and liberating bone marrow cells from the interior of the bone; passing the liberated bone marrow cells through the second tubing; and recovering the liberated bone marrow cells into a collection container.

In various embodiments, the bone is a femur, a tibia, a humerus, an ilium, the sternum, the skull, a rib, or a vertebra (i.e., the vertebral body of the vertebra).

In some embodiments, the bone is in vivo in a living donor, is in vivo in a deceased donor, or is ex vivo in an deceased donor.

In embodiments, creating the first opening and the second opening is performed, respectively, by a first trocar bone marrow needle (or a similar device/instrument suitable for piercing hard cortical bone) and a second trocar bone marrow needle (or a similar device/instrument suitable for piercing hard cortical bone), optionally, wherein the first trocar bone marrow needle and the second trocar bone marrow needle, respectively, comprise the first cannula and the second cannula.

In various embodiments, the method further comprises a step of sealing a gap between the first cannula and the bone surrounding the first opening and/or sealing a gap between the second cannula and the bone surrounding the second opening.

In some embodiments, the second trocar bone marrow needle is round with a single lumen opening or side ports or is flat with a single lumen opening or side ports.

In embodiments, the perfusion medium is passed through the first tubing, the bone, and/or the second tubing via a pressure provided by a pump, a syringe, or by gravity. In various embodiments, the pump is a peristalsis pump, a diaphragm pump, or a syringe pump. In some embodiments, the pressure is provided by gravity where the height of the source of perfusion medium relative to the height of the first cannula regulates the pressure. In embodiments, the pressure is a negative pressure provided by vacuum applied downstream of the bone. In various embodiments, the pressure is a negative pressure provided by vacuum applied downstream of the collection container.

In some embodiments, the first tubing and the second tubing are distinct tubes that are operably linked, e.g., by a third tubing or by a connector.

In embodiments, the source of perfusion buffer is connected to the first tubing. In various embodiments, the first tubing is connected to the source of perfusion buffer via a first valve. In some embodiments, the first valve is selectable to permit release or prevent release of the perfusion medium from its source. In embodiments, the second tubing is connected to the collection container. In various embodiments, the second tubing is connected to the collection container via a second valve. In some embodiments, the second valve is selectable to allow circulation of the perfusion medium from the second tubing to the first tubing or to route the bone marrow cells into the collection container. In embodiments, the first valve is initially selected to permit release of the perfusion medium from its source and the second valve is initially selected to allow circulation of the perfusion medium from the second tubing to the first tubing rather than routing the bone marrow cells into the collection container. In various embodiments, the method further comprises a step of selecting the first valve to prevent release of the perfusion medium from its source while continuing the pressure, thereby allowing circulation of the perfusion medium through first tubing, the bone, and the second tubing. In some embodiments, the circulation of the perfusion medium continues at least until the circulating perfusion medium contains a detectable amount of bone marrow cells, e.g., the perfusion medium becomes turbid with bone marrow cells or cells can be visualized in a sample of perfusion medium through a microscope. In embodiments, the method further comprises a step of selecting the second valve to route the bone marrow cells into the collection container, optionally, selecting the first valve to permit release of the perfusion medium from its source. In some embodiments, the method further comprises a step of selecting the first valve to prevent release of the perfusion medium from its source and selecting the second valve to allow circulation of the perfusion medium from the second tubing to the first tubing rather than routing the bone marrow cells into the collection container.

In various embodiments, the perfusion medium comprises an electrolyte solution and/or a sterile, nonpyrogenic, isotonic solution. In embodiments, the electrolyte solution or the sterile, nonpyrogenic, isotonic solution is a normal saline, a saline buffer (e.g., Ringer's lactate, Tyrode's solution, and PBS), PLASMA-LYTE™, or ISOLYTE®. In some embodiments, the perfusion medium comprises two or more, three or more, or four of a nuclease, human serum albumin (HSA), heparin, and an electrolyte solution. In various embodiments, the perfusion medium further comprises a growth media, e.g., Iscove's Modified Dulbecco's Media (IMDM). In embodiments, the nuclease cleaves both DNA and RNA rather than just DNA (e.g., the nuclease is BENZONASE® or DENARASE®).

In some embodiments, the perfusion medium further comprises an enzyme. In various embodiments, the enzyme comprises a neutral protease, collagenase, extracellular matrix component digestive enzyme, another digestive enzyme, or a combination thereof. In embodiments, the enzyme enhances release of a subset of bone marrow cells.

In some embodiments, the subset of bone marrow cells comprises mesenchymal stromal/stem cells (MSCs). In various embodiments, the MSCs are vertebral bone adherent mesenchymal stromal/stem cells (vBA-MSCs).

In embodiments, the liberated bone marrow cells comprise hematopoietic stem cells (HSCs). In some embodiments, the HSCs are CD34+ cells.

In embodiments, the liberated bone marrow cells comprise mesenchymal stromal/stem cells (MSCs). In various embodiments, the bone is a vertebral body and the MSCs are vertebral bone marrow mesenchymal stromal/stem cells (vBM-MSCs) and/or vertebral bone adherent mesenchymal stromal/stem cells (vBA-MSC).

Another aspect of the present disclosure is a liberated bone marrow cell recovered by any herein-disclosed method.

Yet another aspect of the present disclosure is a system for recovering liberated bone marrow cells. The system comprising a first tubing, a second tubing, a first cannula (optionally, a first trocar bone marrow needle comprising the first cannula), a second cannula (optionally, a second trocar bone marrow needle comprising the second cannula), a source for containing a perfusion medium, a collection container for collecting liberated bone marrow cells, and an apparatus for providing pressure through, the first tubing or the second tubing. In some embodiments, the system further comprises a first valve connecting the first tubing and the source for containing the perfusion medium and/or a second valve connecting the second tubing and the collection container for collecting liberated bone marrow cells.

Any aspect or embodiment described herein can be combined with any other aspect or embodiment as disclosed herein.

### BRIEF DESCRIPTION OF THE DRAWINGS

FIG. 1 illustrates a system for recovering bone marrow cells.

### DETAILED DESCRIPTION

An aspect of the present disclosure is a method for recovering bone marrow cells. The method comprising steps of: creating a first opening at a first surface of a bone comprising bone marrow and creating a second opening at a second surface of the bone; inserting a first cannula through the first opening and inserting a second cannula through the second opening, wherein the first cannula and second cannula gain contact with the interior of the bone; attaching a first tubing to the first cannula and a second tubing to the second cannula; providing a perfusion buffer through the first tubing, thereby perfusing the interior of the bone with the perfusion buffer and liberating bone marrow cells from the interior of the bone; passing the liberated bone marrow cells through the second tubing; and recovering the liberated bone marrow cells into a collection container.

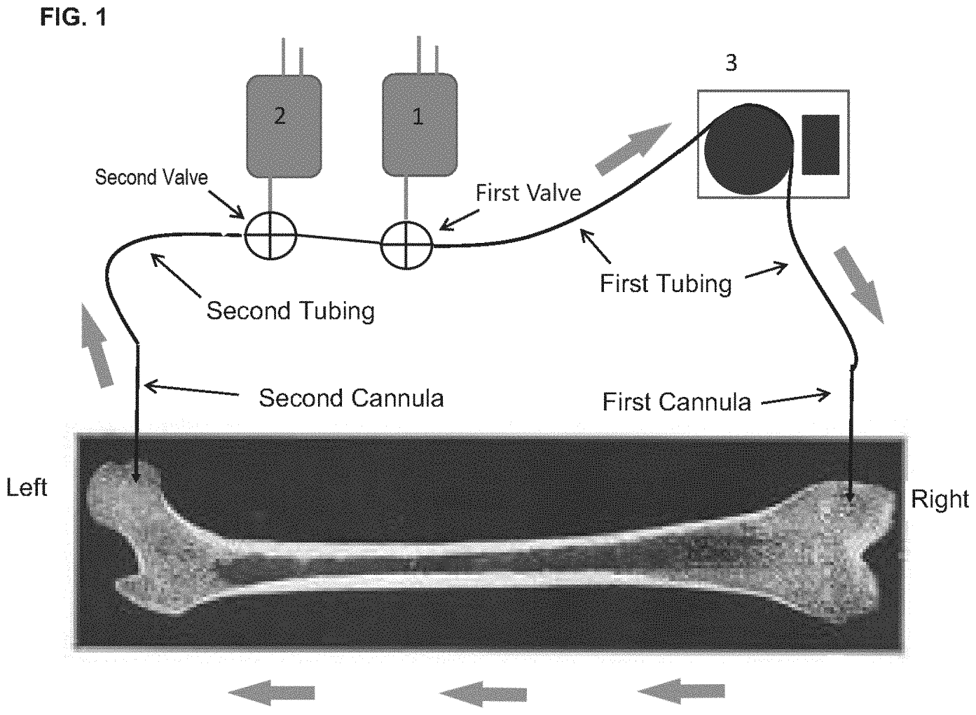

The methods of the present disclosure are illustrated by the system shown in FIG. 1. In the FIGURE, the bone shown is a femur. However, it should be appreciated that any bone comprising bone marrow may be used; examples include a tibia, a humerus, an ilium, the sternum, the skull, a rib, or a vertebra (i.e., the vertebral body of the vertebra). Moreover, the bone may be in vivo in a living donor, in vivo in a deceased donor or may be ex vivo from a deceased donor.

Feature 1 is a source of perfusion buffer. In some embodiments the source may be a bag or a bottle filled with a perfusion buffer. The perfusion buffer is described elsewhere herein; however, the perfusion medium comprises, at least, an electrolyte solution and/or a sterile, nonpyrogenic, isotonic solution; the perfusion medium may comprise enzymes that promote liberation of the bone marrow cells. The source of perfusion buffer is directly or indirectly connected to a first tubing that is directed towards the right end of the bone. Shown in the FIGURE is feature 3—a peristaltic pump which provides pressure that forces the perfusion medium through the first tubing. Additional or alternate pressure-providing apparatuses or methods may be used; also, optimal pressure and/or flow rate of the perfusion medium can be determined depending on the bone type and whether the bone is in vivo or ex vivo. In the FIGURE, the first tubing is connected to the first cannula (black arrow pointing downward) that is inserted into the bone at its right end. The first cannula is passed through a first opening in the bone; the first cannula contacts the interior of the bone, e.g., which comprises bone marrow. At the left end of the bone is a second black arrow that is inserted into the bone. This second arrow represents a second cannula that is passed through a second opening in the bone; the second cannula contacts the interior of the bone, e.g., which comprises bone marrow. Pressure from the peristaltic pump (as an example) forces the perfusion medium through the first tubing and into the interior of the bone, which liberates bone marrow cells from the bone. The liberated bone marrow cells pass through the second cannula and through the second tubing. The second tubing may be directly or indirectly connected to a collection container (feature 2) for recovering the liberated bone marrow cells.

In some embodiments, the first cannula and/or the second cannula independently comprise an array of cannulas. Thus, the first cannula may comprise a plurality of cannulas and/or the second cannula may comprise a plurality of cannulas. Such arrays may increase flow rates and/or may increase liberation of bone marrow cells by contacting the bone marrow at distinct locations.

Flow/circulation of perfusion medium is shown by the large arrows directed clockwise and located at the perimeter of the system. The perfusion medium is circulated through the first tubing, the bone, and the second tubing. A first valve may be selected to prevent release of the perfusion medium from its source and a second valve located distinct from or within an optional manifold may be selected to allow circulation of the perfusion medium from the second tubing to the first tubing rather than routing the bone marrow cells into the collection container. Alternately, independent of the first valve, the flow of perfusion medium from the source may be driven by gravity, with the height of the source relative to the first cannula determining the amount of pressure.

Circulation of the perfusion medium may continue until a sufficient amount of bone marrow cells are liberated, e.g., when the circulating perfusion medium contains a detectable amount of bone marrow cells such as when the perfusion medium becomes turbid with bone marrow cells or cells can be visualized in a sample of perfusion medium through a microscope.

When the circulating perfusion medium comprises a sufficient amount of bone marrow cells, the second valve may be selected to route the liberated bone marrow cells into the collection container with or without selecting the first valve to permit release of the perfusion medium from its source. Alternately, independent of the second valve, the height of the collection bag may be lowered relative to the height of the second cannula such that a negative pressure is applied by the collection container, thereby drawing the perfusion medium comprising liberated bone marrow cells into the collection container.

This illustrative method allows recovery of liberated bone marrow cells with a reduced likelihood of contamination when compared to a standard method, e.g., of repeatedly using a bone marrow aspiration needle and a syringe to draw the marrow from the bone.

### Preparing an In Vivo Donor Bone

A bone to be used in the present method may be present in a living human. Methods for preparing an alive human donor may follow standard protocols used for bone marrow donations. Alternately, the bone may be in vivo in a deceased donor. The standard protocols used for living bone marrow donations may be adapted for in vivo cadaver bones.

The in vivo bone may be any bone suitable for live bone marrow donations, e.g., a femur, a tibia, a humerus, an ilium, the sternum, a rib, or a vertebra (i.e., vertebral body). The vertebral body and the ilium are considered to be the largest consistent reservoirs of high-quality bone marrow.

### Preparing an Ex Vivo Cadaver Bone

Cadaver bones can be procured according to fixed protocols for clinical recovery. Bones can be recovered by surgeons or by personnel at a trained OPO (organ procurement organization) using an osteotome and mallet from consented organ and tissue donors. Unprocessed bones are preferably wrapped in sponges and towels soaked in saline to ensure moisture retention during hypothermic shipment on wet ice at a temperature of 0 to 10° F. to a processing facility.

The process for preparing the donor bone can occur soon after the bone is obtained from the deceased donor or can occur after the donor bone has been shipped in a hypothermic environment to a processing facility.

Since the donor bone can experience prolonged periods of ischemia during recovery and shipment to the processing facility, care must be taken to track the length and type of ischemia—i.e., warm ischemia and cold ischemia. As described in more detail herein, ex vivo cadaver bone subject to predetermined periods of warm and/or cold ischemia are suitable for obtaining meaningful quantities of viable bone marrow cells.

In some embodiments, an ex vivo cadaver bone is not debrided before creating a first opening at a first surface of the bone or creating a second opening at a second surface of the bone.

In some embodiments, the bone is surface sterilized before creating a first opening at a first surface of the bone or creating a second opening at a second surface of the bone. The surface sterilization may comprise contacting the bone with one or both of a bleach solution and a H₂O₂ solution. In some embodiments, the bone is contacted with the bleach solution before contacting the bone with the H₂O₂ solution.

In alternate embodiments, the bone is not surface sterilized before creating a first opening at a first surface of the bone or creating a second opening at a second surface of the bone.

In some embodiment, the ex vivo cadaver bone is freshly obtained and was not frozen prior to creating a first opening at a first surface of the bone or creating a second opening at a second surface of the bone.

In some embodiment, the ex vivo cadaver bone was thawed after being frozen prior to creating a first opening at a first surface of the bone or creating a second opening at a second surface of the bone.

The ex vivo bone may be any bone suitable for bone marrow donations, e.g., a femur, a tibia, a humerus, an ilium, the sternum, the skull, a rib, or a vertebra (i.e., vertebral body). The vertebral body and the ilium are considered to be the largest consistent reservoirs of high-quality bone marrow.

### Perfusion Medium

The perfusion medium comprises, at least, an electrolyte solution and/or a sterile, nonpyrogenic, isotonic solution. In some embodiments, the perfusion medium is merely an electrolyte solution, e.g., a normal saline, a saline buffer (e.g., Ringer's lactate, Tyrode's solution, and PBS), PLASMA-LYTE™, or ISOLYTE®.

The perfusion medium may comprise two or more (e.g., three or more or four) of a nuclease, human serum albumin (HSA), heparin, and an electrolyte solution. Heparin is used as an anticoagulant. HSA provides a protein source to prevent cell adherence and adsorption to surfaces, as well as reactive oxygen scavenging.

The perfusion medium may further comprise a growth media, e.g., Iscove's Modified Dulbecco's Media (IMDM).

The nuclease may be capable of cleaving both DNA and RNA (rather than just DNA, which is the case for DNAse); such enzymes can reduce the viscosity of the solution in which the cells are suspended. Two examples of such an enzyme are BENZONASE® and DENARASE®.

It is noted that IMDM (Iscove's Modified Dulbecco's Media) can substitute for the PLASMA-LYTE™-A, since IMDM is suitable for rapidly proliferating high-density cell cultures and ideal for supporting T- and B-lymphocytes.

The perfusion medium may comprise an enzyme. The enzyme may be one or more of a neutral protease, collagenase, or another digestive enzyme, or a combination thereof which enhances release of bone marrow cells. When the bone is a vertebral body, the enzyme helps liberate vertebral bone adherent mesenchymal stromal/stem cells (vBA-MSCs).

A perfusion solution may comprise ethylenediaminetetraacetic acid (EDTA), e.g., about 2 mM EDTA.

In some embodiments, the amount of heparin in the perfusion medium is about 5 U/ml to about 15 U/ml. In some embodiments, the amount of heparin in the perfusion medium is about 5 U/ml to about 6 U/ml, about 5 U/ml to about 7 U/ml, about 5 U/ml to about 8 U/ml, about 5 U/ml to about 9 U/ml, about 5 U/ml to about 10 U/ml, about 5 U/ml to about 11 U/ml, about 5 U/ml to about 12 U/ml, about 5 U/ml to about 13 U/ml, about 5 U/ml to about 14 U/ml, about 5 U/ml to about 15 U/ml, about 6 U/ml to about 7 U/ml, about 6 U/ml to about 8 U/ml, about 6 U/ml to about 9 U/ml, about 6 U/ml to about 10 U/ml, about 6 U/ml to about 11 U/ml, about 6 U/ml to about 12 U/ml, about 6 U/ml to about 13 U/ml, about 6 U/ml to about 14 U/ml, about 6 U/ml to about 15 U/ml, about 7 U/ml to about 8 U/ml, about 7 U/ml to about 9 U/ml, about 7 U/ml to about 10 U/ml, about 7 U/ml to about 11 U/ml, about 7 U/ml to about 12 U/ml, about 7 U/ml to about 13 U/ml, about 7 U/ml to about 14 U/ml, about 7 U/ml to about 15 U/ml, about 8 U/ml to about 9 U/ml, about 8 U/ml to about 10 U/ml, about 8 U/ml to about 11 U/ml, about 8 U/ml to about 12 U/ml, about 8 U/ml to about 13 U/ml, about 8 U/ml to about 14 U/ml, about 8 U/ml to about 15 U/ml, about 9 U/ml to about 10 U/ml, about 9 U/ml to about 11 U/ml, about 9 U/ml to about 12 U/ml, about 9 U/ml to about 13 U/ml, about 9 U/ml to about 14 U/ml, about 9 U/ml to about 15 U/ml, about 10 U/ml to about 11 U/ml, about 10 U/ml to about 12 U/ml, about 10 U/ml to about 13 U/ml, about 10 U/ml to about 14 U/ml, about 10 U/ml to about 15 U/ml, about 11 U/ml to about 12 U/ml, about 11 U/ml to about 13 U/ml, about 11 U/ml to about 14 U/ml, about 11 U/ml to about 15 U/ml, about 12 U/ml to about 13 U/ml, about 12 U/ml to about 14 U/ml, about 12 U/ml to about 15 U/ml, about 13 U/ml to about 14 U/ml, about 13 U/ml to about 15 U/ml, or about 14 U/ml to about 15 U/ml. In some embodiments, the amount of heparin in the perfusion medium is about 5 U/ml, about 6 U/ml, about 7 U/ml, about 8 U/ml, about 9 U/ml, about 10 U/ml, about 11 U/ml, about 12 U/ml, about 13 U/ml, about 14 U/ml, or about 15 U/ml. In some embodiments, the amount of heparin in the perfusion medium is at least about 5 U/ml, about 6 U/ml, about 7 U/ml, about 8 U/ml, about 9 U/ml, about 10 U/ml, about 11 U/ml, about 12 U/ml, about 13 U/ml, or about 14 U/ml. In some embodiments, the amount of heparin in the perfusion medium is at most about 6 U/ml, about 7 U/ml, about 8 U/ml, about 9 U/ml, about 10 U/ml, about 11 U/ml, about 12 U/ml, about 13 U/ml, about 14 U/ml, or about 15 U/ml.

In some embodiments, the amount of Benzonase® or Denarase® in the perfusion medium is about 11 U/ml to about 55 U/ml. In some embodiments, the amount of Benzonase in the perfusion medium is about 11 U/ml to about 15 U/ml, about 11 U/ml to about 20 U/ml, about 11 U/ml to about 25 U/ml, about 11 U/ml to about 30 U/ml, about 11 U/ml to about 35 U/ml, about 11 U/ml to about 40 U/ml, about 11 U/ml to about 45 U/ml, about 11 U/ml to about 50 U/ml, about 11 U/ml to about 55 U/ml, about 15 U/ml to about 20 U/ml, about 15 U/ml to about 25 U/ml, about 15 U/ml to about 30 U/ml, about 15 U/ml to about 35 U/ml, about 15 U/ml to about 40 U/ml, about 15 U/ml to about 45 U/ml, about 15 U/ml to about 50 U/ml, about 15 U/ml to about 55 U/ml, about 20 U/ml to about 25 U/ml, about 20 U/ml to about 30 U/ml, about 20 U/ml to about 35 U/ml, about 20 U/ml to about 40 U/ml, about 20 U/ml to about 45 U/ml, about 20 U/ml to about 50 U/ml, about 20 U/ml to about 55 U/ml, about 25 U/ml to about 30 U/ml, about 25 U/ml to about 35 U/ml, about 25 U/ml to about 40 U/ml, about 25 U/ml to about 45 U/ml, about 25 U/ml to about 50 U/ml, about 25 U/ml to about 55 U/ml, about 30 U/ml to about 35 U/ml, about 30 U/ml to about 40 U/ml, about 30 U/ml to about 45 U/ml, about 30 U/ml to about 50 U/ml, about 30 U/ml to about 55 U/ml, about 35 U/ml to about 40 U/ml, about 35 U/ml to about 45 U/ml, about 35 U/ml to about 50 U/ml, about 35 U/ml to about 55 U/ml, about 40 U/ml to about 45 U/ml, about 40 U/ml to about 50 U/ml, about 40 U/ml to about 55 U/ml, about 45 U/ml to about 50 U/ml, about 45 U/ml to about 55 U/ml, or about 50 U/ml to about 55 U/ml. In some embodiments, the amount of Benzonase in the perfusion medium is about 11 U/ml, about 15 U/ml, about 20 U/ml, about 25 U/ml, about 30 U/ml, about 35 U/ml, about 40 U/ml, about 45 U/ml, about 50 U/ml, or about 55 U/ml. In some embodiments, the amount of Benzonase in the perfusion medium is at least about 11 U/ml, about 15 U/ml, about 20 U/ml, about 25 U/ml, about 30 U/ml, about 35 U/ml, about 40 U/ml, about 45 U/ml, or about 50 U/ml. In some embodiments, the amount of Benzonase in the perfusion medium is at most about 15 U/ml, about 20 U/ml, about 25 U/ml, about 30 U/ml, about 35 U/ml, about 40 U/ml, about 45 U/ml, about 50 U/ml, or about 55 U/ml.

In some embodiments, the amount of Benzonase® in the perfusion medium is about 1 U/ml to about 10 U/ml. In some embodiments, the amount of Benzonase in the perfusion medium is about 1 U/ml to about 2 U/ml, about 1 U/ml to about 3 U/ml, about 1 U/ml to about 4 U/ml, about 1 U/ml to about 5 U/ml, about 1 U/ml to about 6 U/ml, about 1 U/ml to about 7 U/ml, about 1 U/ml to about 8 U/ml, about 1 U/ml to about 9 U/ml, about 1 U/ml to about 10 U/ml, about 2 U/ml to about 3 U/ml, about 2 U/ml to about 4 U/ml, about 2 U/ml to about 5 U/ml, about 2 U/ml to about 6 U/ml, about 2 U/ml to about 7 U/ml, about 2 U/ml to about 8 U/ml, about 2 U/ml to about 9 U/ml, about 2 U/ml to about 10 U/ml, about 3 U/ml to about 4 U/ml, about 3 U/ml to about 5 U/ml, about 3 U/ml to about 6 U/ml, about 3 U/ml to about 7 U/ml, about 3 U/ml to about 8 U/ml, about 3 U/ml to about 9 U/ml, about 3 U/ml to about 10 U/ml, about 4 U/ml to about 5 U/ml, about 4 U/ml to about 6 U/ml, about 4 U/ml to about 7 U/ml, about 4 U/ml to about 8 U/ml, about 4 U/ml to about 9 U/ml, about 4 U/ml to about 10 U/ml, about 5 U/ml to about 6 U/ml, about 5 U/ml to about 7 U/ml, about 5 U/ml to about 8 U/ml, about 5 U/ml to about 9 U/ml, about 5 U/ml to about 10 U/ml, about 6 U/ml to about 7 U/ml, about 6 U/ml to about 8 U/ml, about 6 U/ml to about 9 U/ml, about 6 U/ml to about 10 U/ml, about 7 U/ml to about 8 U/ml, about 7 U/ml to about 9 U/ml, about 7 U/ml to about 10 U/ml, about 8 U/ml to about 9 U/ml, about 8 U/ml to about 10 U/ml, or about 9 U/ml to about 10 U/ml. In some embodiments, the amount of Benzonase in the perfusion medium is about 1 U/ml, about 2 U/ml, about 3 U/ml, about 4 U/ml, about 5 U/ml, about 6 U/ml, about 7 U/ml, about 8 U/ml, about 9 U/ml, or about 10 U/ml. In some embodiments, the amount of Benzonase in the perfusion medium is at least about 1 U/ml, about 2 U/ml, about 3 U/ml, about 4 U/ml, about 5 U/ml, about 6 U/ml, about 7 U/ml, about 8 U/ml, or about 9 U/ml. In some embodiments, the amount of Benzonase in the perfusion medium is at most about 2 U/ml, about 3 U/ml, about 4 U/ml, about 5 U/ml, about 6 U/ml, about 7 U/ml, about 8 U/ml, about 9 U/ml, or about 10 U/ml.

In some embodiments, HSA is present in the perfusion medium at about 0.5% to about 5%. In some embodiments, HSA is present in the perfusion medium at about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 0.5% to about 4.5%, about 0.5% to about 5%, about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about 5%, about 4% to about 4.5%, about 4% to about 5%, or about 4.5% to about 5%. In some embodiments, HSA is present in the perfusion medium at about 0.5%, about 1%, about 1.5%, about 2%, about 2.5%, about 3%, about 3.5%, about 4%, about 4.5%, or about 5%. In some embodiments, HSA is present in the perfusion medium at least about 0.5%, about 1%, about 1.5%, about 2%, about 2.5%, about 3%, about 3.5%, about 4%, or about 4.5%. In some embodiments, HSA is present in the perfusion medium at most about 1%, about 1.5%, about 2%, about 2.5%, about 3%, about 3.5%, about 4%, about 4.5%, or about 5%.

The perfusion medium may be PLASMA-LYTE™-A as a base with 10 U/mL heparin, 2.5% human serum albumin (HSA), and 3 U/mL of a nuclease that cleaves both DNA and RNA (BENZONASE® or DENARASE®).

Any of the herein-disclosed perfusion media may be used as a wash medium or a resuspension medium.

### Fat Removal and Concentration

The liberated bone marrow cell product recovered from a perfusion system of the present disclosure can be considered to be a fatty emulsion. To prepare a cell product suitable for future use (e.g., administration to a subject in need) it is helpful to reduce or remove the fat content from the liberated bone marrow cell product.

In some embodiments, a collection container (or another container) comprising liberated bone marrow cells is centrifuged to concentrate the collected bone marrow cells. In one embodiment, the containers are centrifuged at 500×g for 15 minutes at room temperature to concentrate the cells. Following the centrifuging the contents of the container form a fat layer overlaying the concentrated bone marrow cells. Thus, in methods of the present disclosure, a step of removing the concentrated bone marrow cells from the fat layer is performed. When the container containing the cell product and which was centrifuged is a bag, a bag clamp or clip is placed on the bag just below the fat layer, to clamp off or squeeze the bag closed beneath the fat layer. The pellet comprising concentrated bone marrow cells can then be drained from the centrifuge bag into the new sterile container, with the bag clip preventing passage of the fat layer. Other methods for separating the fat layer from the concentrated bone marrow cells can be used, e.g., decanting the overlaying fat layer.

An initially de-fatted cell product can be washed, e.g., with fresh perfusion medium, and further de-fatted as necessary.

Cell products can be further washed and concentrated by filtration or centrifugation to obtain a concentrated bone marrow cells.

Filtration is useful for removing any solid matter, e.g., blood vessels and bone particles, that may be present in the cell liberated bone marrow cells.

A sample of the bone marrow cells can be tested by a hematology analyzer, such as a Sysmex Hematology Analyzer, to determine the TNC (total nucleated cell) content of the sample, as an indicator of the TNC content of the bone marrow being subsequently processed.

Cell Isolation from Liberated Bone Marrow Cells

CD34-expressing (CD34+) cells may be isolated from the liberated bone marrow cells. For this, the bone marrow cells are separated using density reduced FICOLL® (a neutral, highly branched, high-mass, hydrophilic polysaccharide which dissolves readily in aqueous solutions) and/or using an immunomagnetic CD34+ cell isolation kit. Cell isolation using density reduced FICOLL® prior to CD34 selection may produce a subset of highly pure and viable CD45/CD34+ cells from bone marrow.

Liberated bone marrow cells, which have been de-fatted, washed, and/or filtered, are concentrated by centrifugation. The following steps may be used with freshly recovered bone marrow cells or bone marrow cells that have been cryopreserved and thawed. In some embodiments, FICOLL® at a density of 1.077 g/mL (the standard/stock density) is used. In alternate embodiments, reduced density FICOLL® is used. To obtain reduced density FICOLL®, FICOLL®-Paque PLUS (density 1.077 g/mL, GE Company) is mixed with PLASMA LYTE™-A Injection pH 7.4 (Baxter Healthcare 2B2544X) in specific proportions to obtain an overall density of less than 1.077 g/ml, particularly 1.063-1.052 g/mL. In one embodiment, the density of 1.063 g/mL is appropriate for isolation of CD34+ cells.

In some embodiments, 5 ml of the 1.063 g/mL density FICOLL® solutions is pipetted into 15-ml centrifuge tubes, and the bone marrow cells (fresh or previously frozen) are carefully layered over the FICOLL® gradient. The tubes are centrifuged for 30 min at 400 g without break at room temperature. After centrifugation, buffy coat cells are harvested carefully, and the cells are washed in phosphate-buffered saline (PBS) containing 0.5% HSA and 2 mM Ethylenediaminetetraacetic acid (EDTA) (MACS buffer, Miltenyi). In some embodiments, centrifugation is performed for 5 min at 400 g, and the resulting cell pellets are resuspended in 10 ml PBS, followed by a second centrifugation for 5 min at 400 g.

Nucleated cells in the isolated buffy coat can be counted using a Sysmex XP-300. A Cellometer Vision (Nexcellom) or flow cytometer can be used to determine cell counts of purified CD34 cells. 20 microliters of AOPI can be added to 20 microliters of cells and after mixing total viable cells can be determined.

In some experiments, in which various FICOLL® densities were tested, it was surprisingly determined that the lower FICOLL® density contemplated in the present disclosure (i.e., 1.063-1.052 gm/mL vs. the conventional 1.077 gm/mL density) leads to more optimum cell recovery. Optimization is based on purity, viability and yield of selected CD34 cells. A target of >90% purity and >90% viable CD34+ cells is preferred. While lower FICOLL® densities resulted in greater purity and fewer dead cells, it was surprisingly found that a greater portion of the CD34+ cells present in the deceased donor whole bone marrow before selection are lost using the lower FICOLL® densities to prepare buffy coat. Thus, the high viability and purity of CD45/CD34+ cells achieved at the conventional FICOLL® density gradient also leads to a large loss in yield (approximately 60% loss of input CD34+ cells).

Alternately or additionally, bone marrow cells expressing CD34+ can be isolated and enriched by contacting the bone marrow cells with the CD34 antibody conjugated with iron, where the bone marrow cells expressing CD34 are then trapped a magnetic separation column (e.g. “CliniMACS®”) (Miltenyi, Bergisch Gladbach, Germany) or an EasySep™ CD34 kit (Stemcell Technologies, Vancouver, BC, Canada) in accordance with the protocol of the manufacturer. The bone marrow cells not expressing CD34 are can be washed away and used to isolate additional subsets of cells present in the bone marrow (e.g., MSCs). The trapped CD34+ bone marrow cells can be harvested by removing the magnetic field and eluting the targeted CD34+ bone marrow cells. Such approach does not require isolating the bone marrow cells with a FICOLL® gradient.

Recovery of MSCs from Liberated Bone Marrow Cells

In another feature of the systems and methods disclosed herein, a method is provided for recovering mesenchymal stem/stromal cells (MSCs) from bone marrow. In some embodiments, MSCs can be obtained from a flow thru solution when CD34+ cells were isolated using a magnetic separation column.

Circulation of the perfusion medium through the bone will liberate one population of MSCs, i.e., the vertebral bone marrow mesenchymal stromal/stem cells (vBM-MSCs), from a vertebral body. Likewise, circulation of the perfusion medium will liberate MSCs from other bone types (e.g., illium and femur). However, another population of MSCs are present in vertebral bodies that resist liberation by circulation of perfusion medium alone. In this method, a perfusion medium comprises a mixture of both collagenase and neutral protease to liberate vertebral bone adherent MSC (vBA-MSC) from vertebral bodies.

In some embodiments, recombinant Clostridium histolyticum collagenase, comprised of the two active isoforms, is used in effective amounts in the MSC liberation process. In some embodiments, the neutral protease may be Paneibacillus polymyxa neutral protease. In various embodiment, the collagenase is DE collagenase (VITACYTE®), which is comprised of purified Clostridium histolyticum collagenase and Paneibacillus polymyxa neutral protease.

Independent of their source bone, freshly liberated MSCs can be characterized by flow cytometry, colony forming unit-fibroblast (CFU-F) potential, population doubling time (PDT) and trilineage (adipogenic, chondrogenic and osteogenic) differentiation in vitro.

In some embodiments, the vBA-MSCs express CD73, CD90, and/or CD105. In various embodiments, the vBA-MSCs possess negligible levels of the hematopoietic stem and progenitor cell surface markers CD14, CD19, CD34 and/or CD45 and/or express very low levels of HLA class II proteins, including human leukocyte antigen DR (HLA-DR).

### Cryopreservation of the Bone Marrow

Bone marrow cells and/or isolated cells obtained therefrom recovered by a herein described method may be cryopreserved for later use.

A freeze media is a solution of a perfusion medium (as disclosed herein) and one or more cryoprotectants. A concentrated bone marrow cell pellet (following centrifugation of liberated bone marrow cells) or concentrated isolated cells (e.g., CD34+ and MSCs) can be combined with a suitable amount of a freeze medium. The amount of freeze medium can be adjusted based upon the cell count in a sample of the cells.

The cryoprotectant can be a permeable media, such as dimethyl sulfoxide (DMSO); 1, 2 propane diol; ethylene glycol; glycerol; foramamide; ethanediol or butane 2, 3 diol; and/or a non-permeable media, such as hydroxyethyl starch (HES), Dextran, sucrose, trehalose, lactose, raffinose, Ribotol, Mannitol or polyvinylpyrrolidone (PVP). From about 2.5% to about 5% HSA also provides cryoprotection through oncotic pressure, cell surface protein stabilization and reactive oxygen scavenging. In a preferred embodiment, the cryoprotectant is DMSO, e.g., in a final concentration of 10%. The perfusion medium can be an electrolyte medium, such as PLASMA-LYTE™, ISOLYTE®, IMDM or other electrolyte solutions suitable for infusion, as disclosed herein elsewhere.

The freeze media can also include concentrations of oxyrase to reduce oxygen content to less than atmospheric, such as to less than 3% of atmospheric concentrations. The addition of oxyrase produces a hypobaric composition that can facilitate cryopreservation.

The freeze media is prepared by mixing the cryoprotectant and the perfusion medium according to the calculated total volume of freeze media needed for the volume of bone marrow cells or isolated cells obtained therefrom collected in the prior steps.

In some embodiments, the freeze media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% HSA. In some embodiments, the freeze media comprises about 1% to about 5% HSA. In some embodiments, the freeze media comprises about 2.5% HSA.

In some embodiments, the freeze media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% DMSO. In some embodiments, the freeze media comprises about 1% to about 10% DMSO. In some embodiments, the freeze media comprises about 5% DMSO.

A bag holding liberated bone marrow cells or isolated cells obtained therefrom (e.g., CD34+ and MSCs) is placed on a rocker for mixing and the freeze media is introduced into the container, e.g., by syringe. The freeze media is introduced at a particular rate over a predetermined time. In one embodiment, the freeze media is added at a rate of 10% of the media per minute, for a time of ten minutes. Once the media has been mixed with the cells, a test sample is extracted by syringe. The remaining mixture of freeze media and cells is injected in predetermined amounts into separate cryopreservation bags. In one embodiment, 70 mL of cells is introduced into a cryopreservation bag and air is drawn out with a syringe. At the end of the process, an 8 mL sample can be removed for sterility testing. Each cryopreservation bag is sealed to create four compartments, which are then separated for storage in cassettes to be stored in a cryo-freezer. In another embodiment, the separated compartments are stored in a passive cooling box, such as a cooling box.

When the test samples from a particular liberated bone marrow cell batch or isolated cells obtained therefrom have been validated for cell count and sterility, the bags of cryopreserved cells can be further cooled for long-term storage. In one embodiment, the bags are cooled at a controlled rate to prevent damage to the cells. In one specific embodiment, the bags are cooled at a rate of −1 to −40° C. per minute to a temperature suitable for plunging the bags into liquid nitrogen. A suitable temperature is in the range of −40 to −100° C. Once that temperature has been reached, the bags are cooled further at a more rapid rate to a temperature of below −130° C. for storage. A cryopreservation bag may be placed within a corresponding compartment of a cooling box and the overlapping cover closed over the compartments to provide a sealed environment for cryopreservation of the contents of the bags. The cooling box is placed within a cryo freezer such that the cooling box produces a cooling rate of −0.5 to −2 C°/min, and typically of −1 C°/min, with nucleation temperatures above −20° C. The freezing process continues at the prescribed rate until the temperature of the cells reaches a suitable temperature. The suitable temperature for storage of the bags is a temperature <−80° C. or <−150° C.

In another embodiment, the bags are cooled in a static chamber temperature as opposed to the controlled rate cryopreservation described above. In the passive cooling approach, the cooling box is placed in a −86° C. freezer until the bags reach a stable temperature.

It is contemplated that the cryopreservation storage can be in many forms. For instance, the cryopreserved cells can be contained in bags of 1 mL to 5 mL volume or vials of 0.1 to 15 mL volumes. In a preferred embodiment, the bags with 70 mL bone marrow are stored in a cooling box within a cryogenic freezer.

The cryopreserved bone marrow cells or isolated cells obtained therefrom is cryobanked for later thawing and extraction of desired cells.

The thawed bone marrow cells or isolated cells obtained therefrom can be provided for a wide range of treatments for human disease and disorders.

The present method provides a system for extracting and banking bone marrow cells and isolated cells obtained therefrom for future clinical use according to the process steps described above. This method can eliminate the failures of the current methods of matching bone marrow donors to groups that are tough to match, such as certain minorities. Once the cells are cryopreserved and banked there is no uncertainty as to the source of the bone marrow, there is no wait for a future recipient and the bone marrow is available in large repeatable volumes.

### Optimizing Cell Viability Based on Ischemia Time

Ischemia time of an ex vivo cadaver donor bone impacts the viability of the bone marrow cells extracted using the processes described herein.

In some embodiments, a method for obtaining bone marrow cells from a cadaver bone comprising at least 80% of the stem cells being viable, comprises steps of measuring warm ischemia time (WIT) and limiting WIT from about one-half to about eight hours; and measuring cold ischemia time (CIT) and limiting CIT from about seven hours to about 40 hours, thereby obtaining at least 80% viable stem cells. In these embodiments, WIT begins at the time of death and ends when the cadaver or cadaver bone is recovered and placed in a cooling environment or condition and CIT begins when the cadaver bone is placed in a cooling environment or condition and ends when processing of the cadaver bone for extraction of cells begins. Here, a relationship exists between the WIT and the CIT, with a higher WIT requiring a lower CIT and a higher CIT requiring a lower WIT. The total ischemia time comprising the sum of WIT and CIT is preferably less than about forty hours. In some embodiments, the Body Cooling Time (BCT) is less than about four hours (e.g., less than about 1 hour or about zero hours); the BCT begins when the cadaver is placed in a cooling environment and ends when cadaver bone is placed in a cooling environment or condition. In some embodiments, the total ischemia time comprising the sum of WIT, CIT, and Body Cooling Time (BCT) is less than about forty hours, with the BCT beginning when the cadaver is placed in a cooling environment and ends when cadaver bone is placed in a cooling environment or condition. Preferably, the BCT is less than one hour, e.g., about zero hours.

Models for determining the effect of ischemia time variables on % CD34+ are described in US 20200399606, the contents of which is incorporated by reference in its entirety. The tables shown in FIGS. 12A-14C of US 20200399606, the contents of which is incorporated by reference in its entirety, can be used to decide whether a particular cadaver bone can yield sufficient cells to warrant further processing of the cadaver bone. In other words, the predictive models can be used to establish ischemia tolerance limits and HSPC quality acceptance criteria. For instance, with respect to the % CD34+ outcome variable, predicted values of over 80% may be required in order to consider the particular donor bone.

The models described above and the examples shown in the tables of FIGS. 12A-14C of US 20200399606 suggest that acceptable levels of HSPC quality are achievable despite the prolonged ischemia times that are inevitable when bones must be procured by geographically-dispersed OPOs and shipped long distances to processing centers. Even under such conditions, favorable combinations of warm- and cold-ischemia times can be achieved, enabling % CD34+ viabilities in the range of 80-90%. The models also suggest that refrigerating the body prior to bone recovery, a practice that is common in the recovery of tissues, is less beneficial in the context of bone marrow recovery. For instance, when whole-body cooling was used, CD34+ viability averaged 72.75%, whereas when body cooling was not used, the average was just under 90%. These models suggest that an optimal practice would be to dispense with body cooling and move recovered bone as quickly as possible to a cold ischemic environment. The models further suggest that limiting WIT (warm ischemia time) to less than eight (8) hours and CIT (cold ischemia time) to less than 40 hours optimizes the opportunity to recover meaningful quantities of viable cells from donor bone.

The models disclosed in US 20200399606 predict viability according to the chart shown in FIG. 15, which shows an 80% CD34+ cell viability threshold is determined to be acceptable. As reflected in the chart, the relationship between warm and cold ischemia times follows a curve from a point at which the WIT is 10 hours and the CIT is 18 hours, to a point at which the WIT is 1 hour and the CIT is 27 hours.

### Methods of Treatment

Liberated bone marrow cells or isolated cells obtained therefrom (e.g., CD34+ and MSCs) of the present disclosure can be provided for a wide range of treatments including treatment for leukemias, brain tumors, breast cancer, Hodgkin's disease, multiple myeloma, neuroblastoma, non-Hodgkin's lymphoma, blood cancers, ovarian cancer, sarcoma, testicular cancer, other solid organ cancer, rheumatoid arthritis, multiple sclerosis, diabetes mellitus, cystic fibrosus, Alzheimer's disease, genetic immunodeficiencies, metabolic disorders, marrow failure syndromes, and HIV. Bone marrow cells can also be used for induction of immunotolerance to reduce the potential rejection of an implant obtained from an organ donor. Bone marrow cell treatments can also be indicated for casualties caused by radiation and certain biological weapons.

Bone marrow is a well-known source for mesenchymal stromal/stem cells (MSCs) which can obtained from herein-disclosed methods. MSCs are self-renewing, multipotent progenitor cells with multilineage potential to differentiate into cell types of mesodermal origin, such as adipocytes, osteocytes, and chondrocytes. In addition, MSCs can migrate to sites of inflammation and exert potent immunosuppressive and anti-inflammatory effects through interactions between lymphocytes associated with both the innate and adaptive immune system. MSCs can be used in treating osteogenesis imperfect, cartilage defects, myocardial infarction, Crohn's disease, multiple sclerosis, autoimmune disease such as Lupus, liver cirrhosis, osteo arthritis, and rheumatoid arthritis. Matched HSC/MSC units which can be used in co-transplant for treatment of graft vs. host disease (GVHD), and for hematopoietic stem cell transplant support.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

As used herein, the term “CD34” is used in reference to an antigen present on immature hematopoietic precursor cells and all hematopoietic colony-forming cells in bone marrow and blood. Certain populations of non-hematopoietic (i.e., CD45 negative) cells also express CD34. Of hematopoietic (i.e., CD45+ cells), the CD34 antigen expression is highest on early progenitor cells and decreases with the maturation of cells. The CD34 antigen is absent on fully differentiated hematopoietic cells. Normal peripheral blood lymphocytes, monocytes, granulocytes, and platelets do not express the CD34 antigen.

The section headings used herein are for organizational purposes only and are not to be construed as limiting the subject matter described.

The present disclosure should be considered as illustrative and not restrictive in character. It is understood that only certain embodiments have been presented and that all changes, modifications and further applications that come within the spirit of the disclosure are desired to be protected.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Example

This example illustrates a system and method of the present disclosure for recovering bone marrow cells. The illustrative system and method are shown in FIG. 1.

In the FIGURE, the bone shown is a femur. However, it should be appreciated that any bone comprising bone marrow may be used; examples include a tibia, a humerus, an ilium, the sternum, the skull, a rib, or a vertebra (i.e., the vertebral body of the vertebra). Moreover, the bone may be in vivo in a living donor, in vivo in a deceased donor or may be ex vivo from a deceased donor.

Feature 1 is a source of perfusion buffer. In some embodiments the source may be a bag or a bottle filled with a perfusion buffer.

The perfusion buffer comprises, at least, an electrolyte solution and/or a sterile, nonpyrogenic, isotonic solution; the perfusion medium may comprise enzymes that promote liberation of the bone marrow cells. The perfusion medium comprises, at least, an electrolyte solution and/or a sterile, nonpyrogenic, isotonic solution. The electrolyte solution or the sterile, nonpyrogenic, isotonic solution may be a normal saline, a saline buffer (e.g., Ringer's lactate, Tyrode's solution, and PBS), PLASMA-LYTE™, or ISOLYTE®. The perfusion medium may comprise two or more, three or more, or four of a nuclease, human serum albumin (HSA), heparin, and an electrolyte solution. The perfusion medium may further comprise a growth media, e.g., Iscove's Modified Dulbecco's Media (IMDM). The nuclease selected cleaves both DNA and RNA rather than just DNA (e.g., the nuclease is BENZONASE® or DENARASE®).

In some cases, the perfusion medium further comprises an enzyme, e.g., a neutral protease, collagenase, extracellular matrix component digestive enzyme, another digestive enzyme, or a combination thereof.

The source of perfusion buffer is directly or indirectly connected to a first tubing that is directed towards the right end of the bone. Shown in the FIGURE is feature 3—a peristaltic pump which provides pressure that forces the perfusion medium through the first tubing. Additional or alternate pressure-providing apparatuses or methods may be used; also, optimal pressure and/or flow rate of the perfusion medium can be determined depending on the bone type and whether the bone is in vivo or ex vivo.

In the FIGURE, the first tubing is connected to the first cannula (black arrow pointing downward) that is inserted into the bone at its right end. The first cannula is passed through a first opening in the bone; the first cannula contacts the interior of the bone, e.g., which comprises bone marrow. At the left end of the bone is a second black arrow that is inserted into the bone. This second arrow represents a second cannula that is passed through a second opening in the bone; the second cannula contacts the interior of the bone, e.g., which comprises bone marrow.

Creating the first opening and the second opening may be performed, respectively, by a first trocar bone marrow needle (or a similar device/instrument suitable for piercing hard cortical bone) and a second trocar bone marrow needle (or a similar device/instrument suitable for piercing hard cortical bone), optionally, the first trocar bone marrow needle and the second trocar bone marrow needle, respectively, comprise the first cannula and the second cannula.

In some cases, the method further comprises a step of sealing a gap between the first cannula and the bone surrounding the first opening and/or sealing a gap between the second cannula and the bone surrounding the second opening.

Pressure from the peristaltic pump (as an example) forces the perfusion medium through the first tubing and into the interior of the bone, which liberates bone marrow cells from the bone. In some cases, perfusion medium is passed through the first tubing, the bone, and/or the second tubing via a pressure provided by a pump, a syringe, or by gravity. In embodiments, the pump is a peristalsis pump, a diaphragm pump, or a syringe pump. The pressure may be provided by gravity where the height of the source of perfusion medium relative to the height of the first cannula regulates the pressure. The pressure may a negative pressure provided by vacuum applied downstream of the bone or the pressure is a negative pressure provided by vacuum applied downstream of the collection container.

The liberated bone marrow cells pass through the second cannula and through the second tubing. The second tubing may be directly or indirectly connected to a collection container (feature 2) for recovering the liberated bone marrow cells.

The first tubing and the second tubing may be distinct tubes that are operably linked, e.g., by a third tubing or by a connector.

In some embodiments, the first cannula and/or the second cannula independently comprise an array of cannulas. Thus, the first cannula may comprise a plurality of cannulas and/or the second cannula may comprise a plurality of cannulas. Such arrays may increase flow rates and/or may increase liberation of bone marrow cells by contacting the bone marrow at distinct locations.

Flow/circulation of perfusion medium is shown by the large arrows directed clockwise and located at the perimeter of the system. The perfusion medium is circulated through the first tubing, the bone, and the second tubing. A first valve may be selected to prevent release of the perfusion medium from its source and a second valve located distinct from or within an optional manifold may be selected to allow circulation of the perfusion medium from the second tubing to the first tubing rather than routing the bone marrow cells into the collection container. Alternately, independent of the first valve, the flow of perfusion medium from the source may be driven by gravity, with the height of the source relative to the first cannula determining the amount of pressure.

Circulation of the perfusion medium may continue until a sufficient amount of bone marrow cells are liberated, e.g., when the circulating perfusion medium contains a detectable amount of bone marrow cells such as when the perfusion medium becomes turbid with bone marrow cells or cells can be visualized in a sample of perfusion medium through a microscope.

When the circulating perfusion medium comprises a sufficient amount of bone marrow cells, the second valve may be selected to route the liberated bone marrow cells into the collection container with or without selecting the first valve to permit release of the perfusion medium from its source. Alternately, independent of the second valve, the height of the collection bag may be lowered relative to the height of the second cannula such that a negative pressure is applied by the collection container, thereby drawing the perfusion medium comprising liberated bone marrow cells into the collection container.

The liberated and recovered bone marrow cells comprise hematopoietic stem cells (HSCs), e.g., CD34+ cells. The liberated and recovered bone marrow cells comprise mesenchymal stromal/stem cells (MSCs). When the bone is a vertebral body, the MSCs are vertebral bone marrow mesenchymal stromal/stem cells (vBM-MSCs) and/or vertebral bone adherent mesenchymal stromal/stem cells (vBA-MSC).

The enzyme added to the perfusion medium release of a subset of bone marrow cells. In some cases, the subset of bone marrow cells comprises mesenchymal stromal/stem cells (MSCs), e.g., vertebral bone adherent mesenchymal stromal/stem cells (vBA-MSCs).

This illustrative method allows recovery of liberated bone marrow cells with a reduced likelihood of contamination when compared to a standard method, e.g., of repeatedly using a bone marrow aspiration needle and a syringe to draw the marrow from the bone.

1. A method for recovering bone marrow cells, the method comprising steps of:
creating a first opening at a first surface of a bone comprising bone marrow and creating a second opening at a second surface of the bone;
inserting a first cannula through the first opening and inserting a second cannula through the second opening, wherein the first cannula and second cannula gain contact with the interior of the bone;
attaching a first tubing to the first cannula and a second tubing to the second cannula;
providing a perfusion buffer through the first tubing, thereby perfusing the interior of the bone with the perfusion buffer and liberating bone marrow cells from the interior of the bone;
passing the liberated bone marrow cells through the second tubing; and
recovering the liberated bone marrow cells into a collection container.

2.-3. (canceled)

4. The method of claim 1, wherein creating the first opening and the second opening is performed, respectively, by a first trocar bone marrow needle (or a similar device/instrument suitable for piercing hard cortical bone) and a second trocar bone marrow needle (or a similar device/instrument suitable for piercing hard cortical bone), optionally, wherein the first trocar bone marrow needle and the second trocar bone marrow needle, respectively, comprise the first cannula and the second cannula.

5. The method of claim 4, further comprising a step of sealing a gap between the first cannula and the bone surrounding the first opening and/or sealing a gap between the second cannula and the bone surrounding the second opening.

6. (canceled)

7. The method of claim 1, wherein the perfusion medium is passed through the first tubing, the bone, and/or the second tubing via a pressure provided by a pump, a syringe, or by gravity.

8. The method of claim 7, wherein the pump is a peristalsis pump, a diaphragm pump, or a syringe pump or wherein the pressure is provided by gravity where the height of the source of perfusion medium relative to the height of the first cannula regulates the pressure, the pressure is a negative pressure provided by vacuum applied downstream of the bone, and/or the pressure is a negative pressure provided by vacuum applied downstream of the collection container.

9.-11. (canceled)

12. The method of claim 1, wherein the first tubing and the second tubing are distinct tubes that are operably linked, e.g., by a third tubing or by a connector.

13. The method of claim 1, wherein a source of perfusion buffer is connected to the first tubing, optionally, wherein the first tubing is connected to the source of perfusion buffer via a first valve.

14. (canceled)

15. The method of claim 13, wherein the first valve is selectable to permit release or prevent release of the perfusion medium from its source.

16. The method of claim 1, wherein the second tubing is connected to the collection container, optionally, wherein the second tubing is connected to the collection container via a second valve.

17. (canceled)

18. The method of claim 16, wherein the second valve is selectable to allow circulation of the perfusion medium from the second tubing to the first tubing or to route the bone marrow cells into the collection container.

19. The method of claim 16, wherein the first valve is initially selected to permit release of the perfusion medium from its source and the second valve is initially selected to allow circulation of the perfusion medium from the second tubing to the first tubing rather than routing the bone marrow cells into the collection container.

20. The method of claim 19, further comprising a step of selecting the first valve to prevent release of the perfusion medium from its source while continuing the pressure, thereby allowing circulation of the perfusion medium through first tubing, the bone, and the second tubing.

21. The method of claim 19, wherein the circulation of the perfusion medium continues at least until the circulating perfusion medium contains a detectable amount of bone marrow cells, e.g., the perfusion medium becomes turbid with bone marrow cells or cells can be visualized in a sample of perfusion medium through a microscope.

22. The method of claim 19, further comprising selecting the second valve to route the bone marrow cells into the collection container, optionally, selecting the first valve to permit release of the perfusion medium from its source.

23. The method of claim 19, further comprising a step of selecting the first valve to prevent release of the perfusion medium from its source and selecting the second valve to allow circulation of the perfusion medium from the second tubing to the first tubing rather than routing the bone marrow cells into the collection container.

24.-25. (canceled)

26. The method of claim 1, wherein the perfusion medium comprises two or more, three or more, or four of a nuclease, human serum albumin (HSA), heparin, and an electrolyte solution.

27. (canceled)

28. The method of claim 26, wherein the nuclease cleaves both DNA and RNA rather than just DNA (e.g., the nuclease is BENZONASE® or DENARASE®).

29.-33. (canceled)

34. The method of claim 1, wherein the liberated bone marrow cells comprise hematopoietic stem cells (HSCs) and/or the liberated bone marrow cells comprise mesenchymal stromal/stem cells (MSCs).

35.-38. (canceled)

39. A system for recovering liberated bone marrow cells, the system comprising a first tubing, a second tubing, a first cannula (optionally, a first trocar bone marrow needle comprising the first cannula), a second cannula (optionally, a second trocar bone marrow needle comprising the second cannula), a source for containing a perfusion medium, a collection container for collecting liberated bone marrow cells, and an apparatus for providing pressure through, the first tubing or the second tubing.

40. The system of claim 39, further comprising a first valve connecting the first tubing and the source for containing the perfusion medium and/or a second valve connecting the second tubing and the collection container for collecting liberated bone marrow cells.
